(19) United States
(12) Patent Application Publication
(10) Pub. No.: US 2008/0248466 A1
(43) Pub. Date: Oct. 9, 2008

(54) Method Of Detecting Mutations In The Gene Encoding Cytochrome P450-2C19

(75) Inventors: James D. Gordon, San Diego, CA (US); Frank Merante, Etobicoke (CA)

Correspondence Address: GOODWIN PROCTER LLP;PATENT ADMINISTRATOR, EXCHANGE PLACE, BOSTON, MA, 02109-2881, US

(73) Assignee: TM BIOSCIENCE PGX INC., Toronto, ON (CA)

(21) Appl. No.: 11/658,798

(22) PCT Filed: Jul. 29, 2005
(86) PCT No.: PCT/CA2005/001180

§ 371 Date: Sep. 12, 2007

Related U.S. Application Data
(60) Provisional application No. 60/592,124, filed on Jul. 30, 2004.

(51) Int. Cl. C12Q 1/68; C07H 21/00

(52) U.S. Cl. 435/6; 536/24.31

(57) ABSTRACT

The present invention describes a method for the simultaneous identification of two or more mutations located in the gene encoding Cytochrome P450-2C19. Multiplex detection is accomplished using multiplexed tagged allele specific primer extension (ASPE) and hybridization of such extended primers to a probe, preferably an addressable anti-tagged support.

### BACKGROUND OF THE INVENTION

1. Field of the Invention

The present invention relates to methods and kits for the detection of mutations located in the gene encoding Cytochrome P450-2C19.

2. Description of the Prior Art

P450-2C19 is a metabolic enzyme involved in the metabolism of a variety of clinically important drugs. Drugs metabolized by P450-2C19 include antidepressants, anxiolytics, antimalarials, and proton pump inhibitors. As with other members of the P450 family, individuals can be classed as extensive metabolizers or poor metabolizers based on the level of 2C19 enzyme activity.

The poor metabolizer phentotype for P450-2C19 is inherited as an autosomal recessive trait. Individuals identified as poor metabolizers using in vivo or in vitro enzyme activity assays have two defective alleles. Presumably heterozygous individuals would have an intermediate level of enzyme activity but this is not resolvable phenotypically using available methods due to the wide inter-individual range in enzyme activity.

Poor metabolizes of P450-2C19 constitute 13 to 23% of Asian populations and 2 to 5% of Caucasians (Desta et al., 2002). The frequency of poor metabolizers has been less extensively studied in African or African American populations but is thought to be roughly 4%. The frequency of poor metabolizers can be as high as 38 to 79% in some islands in Polynesia or Micronesia (Kaneko et al, 1999).

The Gene

The CYP2C19 gene is located on chromosome 10q23 in a cluster of CYP genes including CYP2C8, CYP2C9 and CYP2C18 as well as a number of more distantly related CYP family members. The gene consists of 9 exons spanning a region of roughly 50 Kbp.

Seven of the most common variants in the gene encoding CYP2C19 are listed in Table 1.

TABLE 1
Common Mutations in the Gene Encoding P450-2C19

| Variant | Allele | Defect | Activity | Reference |
|---|---|---|---|---|
| 1A > G | P450- | Loss of | None | Ferguson et al, 1998 |
| | 2C19*4 | initiation codon | | |
| 358T > C | P450- | W120R | None | Ibeanu et al, 1999 |
| | 2C19*8 | | | |
| 395G > A | P450- | R132Q; I331V | None | Ibeanu et al, 1998b |
| | 2C19*6 | | | |
| 636G > A | P450- | New stop codon | None | de Morais et al, |
| | 2C19*3 | | | 1994b |
| 681G > A | P450- | splicing defect | None | de Morais et al, |
| | 2C19*2 | (new) | | 1994a |
| | (A, B) | | | |
| IVS5 + | P450- | splicing defect | None | Ibeanu et al, 1999 |
| 2T > A | 2C19*7 | | | |
| 1297C > T | P450- | R433W | None/ | Xiao et al, 1997 |
| | 2C19*5 | | reduced | Ibeanu et al, 1998a |
| | (A, B) | | | |

P450-2C19 alleles consist of 1 or more nucleotide variants. In each case the haplotypes can be represented by one variant which is unique to that allele and is responsible for the observed phenotype.

Multiplex Allele Specific Primer Extension and Solid Support Detection of Mutations

Multiplex allele specific primer extension, and hybridization of extended primers to a solid support is described generally in the prior art. ASPE technology has been generally described in U.S. Pat. No. 4,851,331. The technology is designed to identify the presence or absence of specific polymorphic sites in the genome.

Multiplex ASPE in conjunction with hybridization to a support for mutation detection can be described generally as follows:

1) Amplifying regions of DNA comprising polymorphic loci utilizing a multiplexed, PCR.

2) Allele specific extension of primers wherein the amplified regions of DNA serve as target sequences for the allele specific extension. Extension primers that possess a 3′ terminal nucleotide which form a perfect match with the target sequence are extended to form extension products. Modified nucleotides are incorporated into the extension product, such nucleotides effectively labelling the extension products for detection purposes. Alternatively, an extension primer may instead comprise a 3′ terminal nucleotide which forms a mismatch with the target sequence. In this instance, primer extension does not occur.

3) Hybridizing the extension product to a probe on a solid support, such as a microarray, wherein the probe is complementary to the 5′ end of the extension product.

The extension primers used in a methodology as described above, possess unique sequence tags at their 5′ ends. For example, the sequence tags may allow the extension products to be captured on a solid support.

Variations of the above technology have been described, for example, in U.S. Pat. No. 6,287,778 and PCT Application (WO 00/47766).

It is an object of the present invention to provide a cost effective, rapid, and accurate method for the detection of variants in the gene encoding P450-2C19.

### SUMMARY OF THE INVENTION

In one embodiment, the present invention provides a method for detecting the presence or absence of variants in a sample selected from the group of mutations identified in Table 1, the method comprising the steps of:

Amplifying regions of DNA which may contain the above mentioned mutations using at least two PCR primer pairs selected from the group of PCR primer pairs consisting of SEQ ID NO: 2 and SEQ ID NO: 3, SEQ ID NO: 4 and SEQ ID NO: 5, SEQ ID NO: 6 and SEQ ID NO: 7, SEQ ID NO: 8 and SEQ ID NO: 9 and SEQ ID NO: 10 and SEQ ID NO: 11.

Hybridizing tagged allele specific extension primers, the allele specific extension primers selected from the group consisting of SEQ ID NO: 12 to SEQ ID NO: 25, to a complementary region of amplified DNA, each tagged allele specific primer having a 3′ portion complementary to a region of the amplified DNA, a 3′ terminal nucleotide complementary to one allele of one of the variant sites (wild type or mutant) mentioned above, and a 5′ portion complementary to a probe (anti-tag) sequence.

Extending tagged ASPE primers, whereby a labelled extension product of the primer is synthesised when the 3′ terminal nucleotide of the primer is complementary to a corresponding nucleotide in the target sequence; no extension product is synthesised when the terminal nucleotide of the primer is not complementary to the corresponding nucleotide in the target sequence.

Hybridizing extension products to a probe and detection of labelled extension products. Detection of a labelled extension product is indicative of the presence of the allele complementary to the 3′-terminal nucleotide of the ASPE primer. In the absence of a labelled extension product, it is determined that the allele corresponding to the 3′ end of the ASPE primer is not present in the sample.

In another embodiment, the present invention provides a kit for use in detecting the presence or absence of at least two variants of the gene encoding P450-2C19 identified in Table 1. The kit comprises at least two tagged allele specific extension primers selected from the group consisting of SEQ ID NO: 10 to SEQ ID NO: 19, and two pcr primer pairs selected from the group consisting of SEQ ID NO: 2 and SEQ ID NO: 3, SEQ ID NO: 4 and SEQ ID NO: 5, SEQ ID NO: 6 and SEQ ID NO: 7, and SEQ ID NO: 8 and SEQ ID NO: 9, and SEQ ID NO: 10 and SEQ ID NO: 11.

### BRIEF DESCRIPTION OF THE DRAWINGS

These and other features of the preferred embodiments of the invention will become more apparent in the following detailed description in which reference is made to the appended drawings wherein:

FIG. 1 depicts a reaction scheme according to one aspect of the method of the present invention.

FIG. 2 depicts the genotyping of an individual having a P450-2C19*2 heterozygous genotype.

FIG. 3 depicts the genotyping of an individual having a 2C19*8 heterozygous genotype.

FIG. 4 depicts the genotyping of an individual having a 2C19*2/*3 compound heterozygous genotype.

### DESCRIPTION OF THE PREFERRED EMBODIMENTS

The following terms used in the present application will be understood to have the meanings defined below.

The terms “oligonucleotide” and “polynucleotide” as used in the present application refer to DNA sequences being of greater than one nucleotide in length. Such sequences may exist in either single or double-stranded form. Examples of oligonucleotides described herein include PCR primers, ASPE primers, and anti-tags.

The term “allele” is used herein to refer to different versions of a nucleotide sequence.

The expression “allele specific primer extension (ASPE)”, as used herein, refers to a mutation detection method utilizing primers which hybridize to a corresponding DNA sequence and which are extended depending on the successful hybridization of the 3′ terminal nucleotide of such primer. Amplified regions of DNA serve as target sequences for ASPE primers. Extension primers that possess a 3′ terminal nucleotide which form a perfect match with the target sequence are extended to form extension products. Modified nucleotides can be incorporated into the extension product, such nucleotides effectively labelling the extension products for detection purposes. Alternatively, an extension primer may instead comprise a 3′ terminal nucleotide which forms a mismatch with the target sequence. In this instance, primer extension does not occur unless the polymerase used for extension inadvertently possesses exonuclease activity.

The term “genotype” refers to the genetic constitution of an organism. More specifically, the term refers to the identity of alleles present in an individual. “Genotyping” of an individual or a DNA sample refers to identifying the nature, in terms of nucleotide base, of the two alleles possessed by an individual at a known polymorphic site.

The term “polymorphism”, as used herein, refers to the coexistence of more than one form of a gene or portion thereof.

The term “PCR”, as used herein, refers to the polymerase chain reaction. PCR is a method of amplifying a DNA base sequence using a heat stable polymerase and a pair of primers, one primer complementary to the (+)-strand at one end of the sequence to be amplified and the other primer complementary to the (−) strand at the other end of the sequence to be amplified. Newly synthesized DNA strands can subsequently serve as templates for the same primer sequences and successive rounds of heat denaturation, primer annealing and strand elongation results in rapid and highly specific amplification of the desired sequence. PCR can be used to detect the existence of a defined sequence in a DNA sample.

The term “primer”, as used herein, refers to a short single-stranded oligonucleotide capable of hybridizing to a complementary sequence in a DNA sample. A primer serves as an initiation point for template dependent DNA synthesis. Deoxyribonucleotides can be joined to a primer by a DNA polymerase. A “primer pair” or “primer set” refers to a set of primers including a 5′ upstream primer that hybridizes with the complement of the 5′ end of the DNA sequence to be amplified and a 3′ downstream primer that hybridizes with the 3′ end of the DNA sequence to be amplified. The term “PCR primer” as used herein refers to a primer used for a PCR reaction. The term “ASPE primer” as used herein refers to a primer used for an ASPE reaction.

The term “tag” as used herein refers to an oligonucleotide sequence that is coupled to an ASPE primer. The sequence is generally unique and non-complementary to the human genome while being substantially complementary to a probe sequence. The probe sequence may be, for example, attached to a solid support Tags serve to bind the ASPE primers to a probe.

The term “tagged ASPE primer” as used herein refers to an ASPE primer that is coupled to a tag.

The term “anti-tag” or “probe” as used herein refers to an oligonucleotide sequence having a sequence complementary to, and capable of hybridizing to, the tag sequence of an ASPE primer. The “anti-tag” may be coupled to a support.

The term “wild type” or “wt” as used herein refers to the normal, or non-mutated, or functional form of a gene.

The term “homozygous wild-type” as used herein refers to an individual possessing two copies of the same allele, such allele characterized as being the normal and functional form of a gene.

The term “heterozygous” or “HET” as used herein refers to an individual possessing two different alleles of the same gene.

The term “homozygous mutant” as used herein refers to an individual possessing two copies of the same allele, such allele characterized as the mutant form of a gene.

The term “mutant” as used herein refers to a mutated, or potentially non-functional form of a gene.

The present invention was developed in response to a need for a rapid, highly specific, and cost-effective method to genotype individuals susceptible to adverse drug reactions. More specifically, the present invention provides a method for identifying individuals who may have drug metabolism defects resulting from mutations in the P450-2C19 gene.

The present invention provides a novel, multiplex method of detecting multiple mutations located in the gene encoding CYP2C19. Specifically, the methodology can be used for the detection of the presence or absence of mutations selected from the group consisting of the variants identified in Table 1. In a preferred embodiment, the present invention provides a method of detecting the presence or absence of all the variants identified in Table 1.

The positive detection of one or more of the variants identified in Table 1 may be indicative of an individual having a predisposition to compromised enzyme activity.

The present invention is further characterized by a high level of specificity. Such specificity is required in order to ensure that any result generated is a true representation of the genomic target and not simply the result of non-specific interactions occurring between reagents present in reactions. This is especially important for multiplexed DNA-based tests where the numerous sequences present in the reaction mixture, most of which are non-complementary, may interact non-specifically depending on the reaction conditions. The ASPE primer and PCR primer sequences described below have been selected due to their minimal cross-reactivity.

The present invention is also characterized by its high level of accuracy when compared to existing methodologies for the detection of mutations in the gene encoding CYP2C19.

The methodology of the present invention utilizes the combination of multiplex ASPE technology with hybridization of tagged and labelled extension products to probes in order to facilitate detection. Such methodology is suitable for high-throughput clinical genotyping applications.

In one embodiment, the present invention provides a method for detecting the presence or absence of variants in a sample selected from the group of mutations identified in Table 1, the method comprising the steps of:

Amplifying regions of DNA which may contain the above mentioned variants.

Hybridizing at least two tagged allele specific extension primers to a complementary region of amplified DNA, each tagged allele specific primer having a 3′ portion complementary to a region of the amplified DNA, a 3′ terminal nucleotide complementary to one allele of one of the mutation sites (wild type or mutant) mentioned above, and a 5′ portion complementary to a probe sequence.

Extending tagged ASPE primers, whereby a labelled extension product of the primer is synthesised when the 3′ terminal nucleotide of the primer is complementary to a corresponding nucleotide in the target sequence; no extension product is synthesised when the terminal nucleotide of the primer is not complementary to the corresponding nucleotide in the target sequence.

Hybridizing extension products to a probe and detection of labelled extension products. Detection of a labelled extension product is indicative of the presence of the allele complementary to the 3 ′-terminal nucleotide of the ASPE primer. In the absence of a labelled extension product, it is determined that the allele corresponding to the 3′ end of the ASPE primer is not present in the sample.

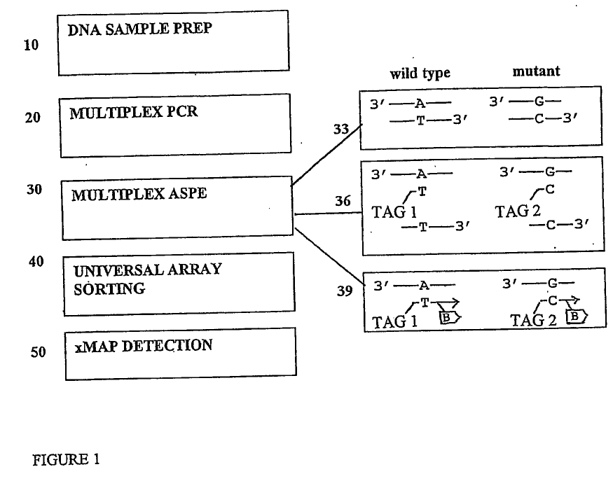

A general overview of the above-mentioned method is presented in FIG. 1. A DNA sample is first prepared 10 using methods known in the art. Multiplex PCR amplification 20 is conducted in order amplify regions of DNA containing variant sites in the gene encoding cytochrome P450-2D6. A multiplex ASPE reaction 30 is then conducted. By example only, 33 illustrates a wild type and a mutant allele of a gene. At step 36 ASPE primers are hybridized to amplified regions of DNA. If the 3′ terminal nucleotide of an ASPE primer is complementary to a corresponding nucleotide in the target sequence, a labelled extension product is formed 39 as will be described further below. The ASPE may be sorted on an addressable universal sorting array 40 wherein the presence of a labelled extension product may be detected using, for example, xMAP detection 50.

### DNA Sample Preparation

Patient samples can be extracted with a variety of methods known in the art to provide nucleic acid (most preferably genomic DNA) for use in the following method. In a preferred embodiment, a DNA sample is extracted from whole blood.

### Amplification

In a first step at least two regions of DNA from the gene encoding CYP2C19 containing variant sites are amplified.

In a preferred embodiment of the present invention, PCR amplification of regions containing variant sites in the gene encoding CYP2C19 is initiated using at least two pairs of PCR primers selected from the group of primer pairs consisting of: SEQ ID NO: 2 and SEQ ID NO: 3, SEQ ID NO: 4 and SEQ ID NO: 5, SEQ ID NO: 6 and SEQ ID NO: 7, SEQ ID NO: 8 and SEQ ID NO: 9 and SEQ ID NO: 10 and SEQ ID NO: 11.

The relationships of each pair of primers to the mutation sites listed in Table 1 is presented in Table 2.

TABLE 2
Primer Pairs Used to Amplify Regions
Containing CYP2C19 Mutations

| PCR Primer Pair | Variants present in Amplimer |
|---|---|
| SEQ ID NO: 2 and 3 | CYP2C19*4 |
| SEQ ID NO: 4 and 5 | CYP2C19*6 and CYP2C19*8 |
| SEQ ID NO: 6 and 7 | CYP2C19*3 |
| SEQ ID NO: 8 and 9 | CYP2C19*2 and CYP2C19*7 |
| SEQ ID NO: 10 and 11 | CYP2C19*5 |

An individual skilled in the art will recognize that alternate PCR primers could be used to amplify the target polymorphic regions, and deletion and duplication regions, however, in a preferred embodiment the primers listed in Table 2 are selected due to their minimal non-specific interaction with other sequences in the reaction mixture.

### ASPE

The ASPE step of the method of the present invention is conducted using tagged ASPE primers selected from the group of ASPE primers consisting of SEQ ID NO: 12 to SEQ ID NO: 25.

The ASPE primer set of the present invention has been optimized to ensure high specificity and accuracy of diagnostic tests utilizing such allele specific primers.

Table 3 presents a listing of the ASPE primers used in a preferred embodiment of the present invention. The suffix “wt” indicates an ASPE primer used to detect the wild type form of the gene encoding CYP2C19 at a specific variant site. The suffix “mut” indicates an ASPE primer used to detect a mutant form of the gene encoding CYP2C 19 at a specific variant site. Bases 1 to 24 of each of SEQ ID NO: 12 to SEQ ID NO: 25 are the 5′ portions of the ASPE primers that are complementary to specific probe sequences. Although the specific sequences listed in Table 2 are preferred, in alternate embodiments of the present invention, it is possible to combine different 5′ portions of the sequences in Table 3 (bases 1 to 24 of SEQ ID NOs: 12 to 25) with different 3′ end hybridizing portions of the sequences in Table 2 (bases 25 and up of SEQ ID NOs: 12 to 25).

The ASPE primers of the present invention are listed in Table 3.

TABLE 3
P450-2C19 ASPE Primer Sequences

| SEQ ID | | |
|---|---|---|
| NO: | Allele Detected | Direction |
| 12 | 2C19*2WT | Reverse |
| 13 | 2C19*2Mut | Reverse |
| 14 | 2C19*3WT | Forward |
| 15 | 2C19*3Mut | Forward |
| 16 | 2C19*4WT | Reverse |
| 17 | 2C19*4Mut | Reverse |
| 18 | 2C19*5WT | Reverse |
| 19 | 2C19*5Mut | Reverse |
| 20 | 2C19*6WT | Reverse |
| 21 | 2C19*6mut | Reverse |
| 22 | 2C19*7WT | Forward |
| 23 | 2C19*7Mut | Forward |
| 24 | 2C19*8WT | Reverse |
| 25 | 2C19*8Mut | Reverse |

The 3′ end hybridizing portion of the extension primer is hybridized to the amplified material. Where the 3′ terminal nucleotide of an ASPE primer is complementary to the polymorphic site, primer extension is carried out using a modified nucleotide. Where the 3′ terminal nucleotide of the ASPE primer is not complementary to the polymorphic region, no primer extension occurs.

In one embodiment, labelling of the extension products is accomplished through the incorporation of biotinylated nucleotides into the extension product which may be identified using fluorescent (Streptavidin-Phycoerythrin) or chemiluminescent (Streptavidin-Horseradish Peroxidase) reactions. However, an individual skilled in the art will recognize that other labelling techniques may be utilized. Examples of labels useful for detection include but are not limited to radiolabels, fluorescent labels (e.g fluorescein and rhodamine), nuclear magnetic resonance active labels, positron emitting isotopes detectable by a positron emission tomography (“PET”) scanner, and chemiluminescers such as luciferin, and enzymatic markers such as peroxidase or phosphatase.

Each ASPE primer used in the methodology as described above, possess a unique sequence tag at their 5′ ends. The sequence tags allow extension products to be detected with a high degree of specificity, for example, through capture on a solid support in order to facilitate detection.

Detection

The tagged 5′ portions of the allele specific primers of the present invention are complementary to probe sequences. Upon hybridization of the allele specific primers to a corresponding probe sequence the presence of extension products can be detected.

In a preferred embodiment, probes used in the methodology of the present invention are coupled to a solid support, for example a ‘universal’ bead-based microarray.

Examples of supports that can be used in the present invention include, but are not limited to, bead based microarrays and 2D glass microarrays. The preparation, use, and analysis of microarrays are well known to persons skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application W095/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.). Detection can be achieved through arrays using, for example, chemiluminescence or fluorescence technology for identifying the presence or absence of specific mutations.

Universal arrays function as sorting tools indirectly detecting the target of interest and are designed to be isothermal and minimally cross-hybridizing as a set. Examples of microarrays which can be used in the present invention include, but should not be limited to, Luminex's® bead based microarray systems, and Metrigenix's™ Flow Thru chip technology.

In one embodiment, for example, Luminex's 100 XMAP™ fluorescence based solid support microarray system is utilized. Anti-tag sequences complementary to the tag regions of the ASPE primers/extension products, described above, are coupled to the surface of internally fluorochrome-color-coded microspheres. An array of anti-tag microspheres is produced, each set of microspheres having its own characteristic spectral address. The mixture of tagged, extended, biotinylated ASPE primers is combined with the array of anti tagged microspheres and is allowed to hybridize under stringent conditions.

In a reaction mixture, a fluorescent reporter molecule (e.g. streptavidin-phycoerythrin) is used to detect labelled extension products which are synthesized when the terminal nucleotide of an ASPE primer is complementary to a corresponding nucleotide in the target sequence.

The reaction mixture, comprising microspheres, extension products etc. is injected into a reading instrument, for example Luminex's 100 xMAP™, which uses microfluidics to align the microspheres in single file. Lasers are used to illuminate the colors both internal to the microspheres, and attached to the surface in the form of extension products hybridized to anti-tag sequences. The Luminex 100 xMAP™, interprets the signal received and identifies the presence of wild type and/or mutant alleles. The presence of the mutant allele of any one or more of the mutations presented in Table 1 may be indicative a predisposition for adverse drug reactions. Software can be provided which is designed to analyze data associated with the specific extension products and anti-tagged microspheres of the present invention.

In another embodiment, the Metrigenix Flow-Thru three dimensional microchannel bio chip (Cheek, B. J., Steel A. B., Torres, M. P., Yu, Y., and Yang H. Anal. Chem. 2001,73,5777-5783) is utilized for genotyping as known in the art. In this embodiment, each set of microchannels represents a different universal anti-tag population. Anti-tag sequences corresponding to the tag regions of the ASPE primers/extension products, described above, are attached to the inner surface of multiple microchannels comprising a cell. Multiple cells make up a chip. The reaction mixture, including biotinylated extension products flows through the cells in the presence of a chemiluminescent reporter substrate such as streptavidin-horseradish peroxidase. Microarray chips can be imaged using technology known in the art, such as an ORCA-ER CCD (Hamamatsu Photonics K. K., Hamamatsu City, Japan), and imaging software, in order to identify the genotype of an individual.

### Kits

In an additional embodiment, the present invention provides kits for the multiplex detection of mutations in the gene encoding CYP2C19.

A kit that can be used for detection of the mutations of interest may contain the following components including: a PCR primer mix for amplifying regions containing mutation sites of interest (optionally including dNTPs), an ASPE primer mix for generation of labelled extension products (optionally including dNTPs) and a solid support, such as microarray beads, the beads having anti-tags complementary to the tagged regions of the ASPE primers. In addition, an individual skilled in the art would recognize other components which could be included in such kits including, for example, buffers and polymerases.

Kits of the present invention may include PCR primer pairs, ASPE primers, and tagged supports for all the mutations to be detected, or may be customized to best suit the needs of an individual end user. For example, if an end user wishes to determine the presence or absence of only four of the mutations in the CYP2C19 gene, a kit can be customized to include only the PCR primer pairs, ASPE primers, and support required for the detection of the desired mutations. As such, the end user of the product can design a kit to match their specific requirements. In addition, the end user can also control the tests to be conducted at the software level when using, for example, a universal bead based-microarray for detection. For example, software can be provided with a kit, such software reading only the beads for the desired mutations or reporting only the results from the desired mutation data. Similar control of data reporting by software can be obtained when the assay is performed on alternate platforms.

An individual skilled in the art will recognize that although the present method has been described in relation to the specific mutations identified in Table 1, PCR primers and ASPE primers used to detect additional mutations could be included in the above method and kits.

### EXAMPLE 1

### ASPE/Microarray Detection of Mutations in the Gene Encoding CYP2C19

1) Oligonucleotides

All oligonucleotides were synthesized by Integrated DNA Technologies (Coralville, Iowa). PCR primers were unmodified and were purified by standard desalting procedures. Universal anti-tags (probes) were 3′-C₇ amino-modified for coupling to carboxylated microspheres. All anti-tags were reverse phase HPLC-purified. Chimeric ASPE primers which consisted of a 24 mer universal tag sequence 5′ to the allele-specific sequence were also unmodified but were purified by polyacrylamide gel electrophoresis. Following reconstitution, exact oligonucleotide concentrations were determined spectrophotometrically using extinction coefficients provided by the supplier. Reconstituted oligonucleotides were scanned between 200 and 800 nm and absorbance was measured at 260 nm to calculate oligonucleotide concentration.

2) Reagents

Platinum Taq, Platinum Tsp, individual dNTPs and biotin-dCTP were purchased from Invitrogen Corporation (Carlsbad, Calif.). Shrimp alkaline phosphatase and exonuclease I were purchased from USB Corporation (Cleveland, Ohio). Carboxylated fluorescent microspheres were provided by Luminex Corporation (Austin, Tex.). The EDC cross-linker (1-ethyl-3-(3-dimethylaminopropyl) carbodiimide hydrochloride) was purchased from Pierce (Rockford, Ill.). OmniPur reagents including MES (2-N-morpholino)ethane sulfonic acid), 10% SDS, NaCl, Tris, Triton X-100, Tween-20 and TE buffer were purchased from EM Science (Darmstadt, Germany). The streptavidin-conjugated phycoerythrin was obtained from Molecular Probes Inc. (Eugene, Oreg.).

3) Genotyping

a) MULTIPLEX PCR (5-plex): Multiplex PCR was carried out using 25 ng genomic DNA in a 25 uL final volume: A ‘no target’ PCR negative control was included with each assay run. The reaction consisted of 20 mmol/L Tris-HCl, pH 8.4, 50 mmol/L KCl, 2.5 mmol/L MgCl2, 200 umol/L each dNTP, 5 units Platinum Taq and primers at 150 nmol/L. Samples were cycled in an MJ Research PTC-200 thermocycler (Waterdowvn, Mass.) with cycling parameters set at 95 ° C. for 5 minutes followed by 30 cycles at 95° C. for 30 seconds, 58° C. for 30 seconds and 72° C. for 30 seconds. Samples were then held at 72° C. for 5 minutes and kept at 4° C. until use.

b) ALLELE-SPECIFIC PRIMER EXTENSION: Prior to the ASPE reaction, each PCR reaction was treated with shrimp alkaline phosphatase (SAP) to inactivate any remaining nucleotides (particularly dCTP) so that biotin-dCTP could be efficiently incorporated during the primer extension reaction. Each PCR reaction was also treated with exonuclease I (EXO) to degrade remaining PCR primers in order to avoid any interference with the tagged ASPE primers and the extension reaction itself. To each 25 uL PCR reaction, 2.0 uL SAP (2.0 units) and 0.5 uL EXO (5 units) were added directly and the sample was vortexed and briefly centrifuged. Samples were then incubated at 37° C. for 30 minutes followed by a 15 minute incubation at 99° C. to inactivate the enzymes. Samples were then added directly to the ASPE reaction.

Multiplex ASPE was carried out using 5 uL of treated PCR product in a final volume of 20 uL. Each reaction consisted of 20 mmol/L Tris-HCl pH 8.4, 50 mmol/L KCl, 1.25 mmol/L MgCl2, 5 umol/L biotin-dCTP, 5 umol/L each of dATP, dGTP and dTTP, 1.5 units Platinum Tsp and 25 nmol/L ASPE primer pool. The ASPE reactions were incubated at 96° C. for 2 minutes and then subjected to 40 cycles at 94° C. for 30 seconds, 52° C. for 30 seconds and 74° C. for 60 seconds. Reactions were then held at 4° C. until use.

c) BEAD COUPLING: Amino-modified anti-tag sequences were coupled to carboxylated microspheres following Luminex's one-step carbodiimide coupling procedure. Briefly, 5×10⁶ microspheres were combined with 1 nmol NH₂-oligo in a final volume of 50 uL 0.1 mol/L MES, pH4.5. A 10 mg/mL EDC working solution was prepared just prior to use and 2.5 uL was added to the bead mixture and incubated for 30 minutes. A second 2.5 uL aliquot of freshly prepared EDC was added followed by an additional 30 minute incubation. Following washes in 0.02% (v/v) Tween-20 and 0.1% (w/v) SDS, the anti-tag coupled beads were resuspended in 100 uL TE buffer (10 mmol/L Tris, pH 8.0, 1 mmol/L EDTA). Bead concentrations were determined using a Beckman Coulter Z2 Particle Count and Size Analyzer (Coulter Corp, Miami Fla.).

d) UNIVERSAL ARRAY HYBRIDIZATION: Each hybridization reaction was carried out using approximately 2500 beads of each of the 10 anti-tag bearing bead populations. The beads were combined in hybridization buffer (0.22 mol/L NaCl, 0.11 mol/L Tris, pH 8.0 and 0.088% (v/v) Triton X-100) and 45 uL of the mix were added to each well of an MJ Research 96-well plate (Reno, Nev.). A 5 uL aliquot of each ASPE reaction was then added directly to each well. The samples were then heated to 96° C. for 2 minutes in an MJ Research PTC-100 followed by a one hour incubation at 37° C. Following this incubation, samples were filtered through a 1.2 um Durapore Membrane (Millipore Corp, Bedford, Mass.) and washed once using wash buffer (0.2 mol/L NaCl, 0.1 mol/L Tris, pH 8.0 and 0.08% (v/v) Triton X-100). The beads were then resuspended in 150 uL reporter solution (1 ug/mL streptavidin-conjugated phycoerythrin in wash buffer) and incubated for 15 minutes at room temperature. The reactions were read on the Luminex xMAP. Acquisition parameters were set to measure 100 events per bead population and a 100 uL sample volume. A gate setting was established prior to running the samples and maintained throughout the course of the study.

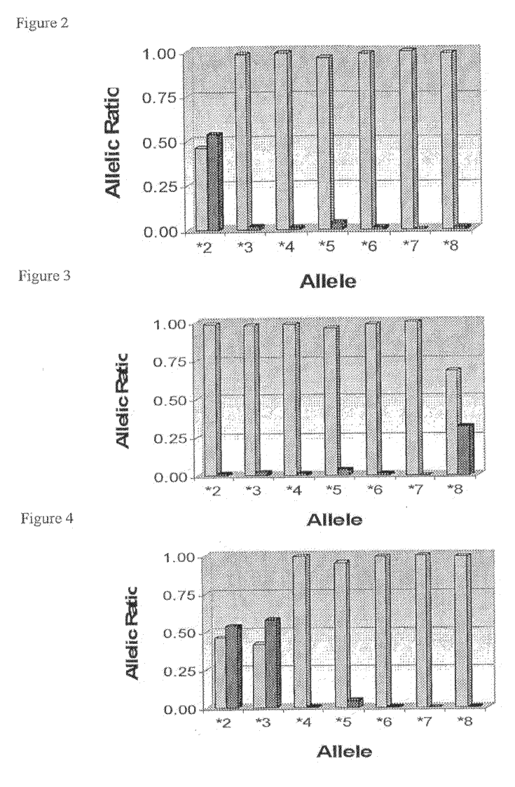

FIGS. 2 to 4 depict a examples of results obtained for samples from different individuals using the method of the present invention. FIG. 2 depicts the genotyping of an individual having a P450-2C19*2 heterozygote genotype. FIG. 3 depicts the genotyping of an individual having a 2C19*8 heterozygote genotype. FIG. 4 depicts the genotyping of an individual having a 2C19*2/*3 compound heterozygote genotype. Data for wild type and mutant alleles is expressed as an Allelic Ratio (i.e., the net signal for the particular variant over the net signal for all variants at a specific position).

All publications, patents and patent applications are herein incorporated by reference in their entirety to the same extent as if each individual publication, patent or patent application was specifically and individually indicated to be incorporated by reference in its entirety.

Although the invention has been described with reference to certain specific embodiments, various modifications thereof will be apparent to those skilled in the art without departing from the spirit and scope of the invention as outlined in the claims appended hereto.

1. A method for detecting in a sample the presence or absence of nucleotide variants at polymorphic sites in the gene encoding cytochrome P450-2C19, said variants selected from the group consisting CYP2C19*2, CYP2C19*3, CYP2C19*4, CYP2C19*5, CYP2C19*6, CYP2C19*7 and CYP2C19*8, the method comprising the steps of;
a) amplifying from the sample regions of DNA containing the variants to form amplified DNA products;
b) hybridizing at least two tagged allele specific extension primers to a complementary target sequence in the amplified DNA products, wherein each tagged allele specific extension primer has a 3′-end hybridizing portion capable of hybridizing to the amplified DNA, and wherein the 3′ end hybridizing portion portions of the at least two tagged allele specific extension primers each comprise a sequence selected from the group consisting of nucleotides from position 25 to the 3′ terminal nucleotide of SEQ ID NO: 12 to SEQ ID NO: 25, and a 5′-end tag portion complementary to a corresponding anti-tag sequence, the terminal nucleotide of the 3′ end hybridizing portion being either complementary to a suspected variant nucleotide or to the corresponding wild type nucleotide of the site;
c) extending the at least two tagged allele specific extension primers, using labelled nucleotides, if the terminal nucleotide of the 3′ end hybridizing portion is a perfect match to an allele of one of the polymorphic sites in the amplified DNA products;
d) hybridizing the at least two tagged allele specific extension primers to the corresponding anti-tag sequence and detecting the presence of labelled extension products.

2. The method of claim 1 wherein the 5′-end tag portions of the at least two tagged allele specific primers each comprise comprises a sequence selected from the group consisting of bases nucleotides from position 1 to 24 of SEQ ID NO: 12 to SEQ ID NO: 25 and wherein the sequence of each 5′-end tag portion is unique.

3. The method of claim 1 wherein the anti-tag sequence is coupled to a solid support.

4. The method of claim 3 wherein the solid support is selected from the group consisting of beads, spectrally coded beads, and a chip based microarray.

5. The method of claim 1 wherein the step of amplifying is conducted by PCR using a set of PCR amplification primers selected from the group of primer pairs consisting of:
SEQ ID NO: 2 and SEQ ID NO: 3, SEQ ID NO: 4 and SEQ ID NO: 5, SEQ ID NO: 6 and SEQ ID NO: 7, SEQ ID NO: 8 and SEQ ID NO: 9 and SEQ ID NO: 10 and SEQ ID NO: 11, wherein the PCR primer pairs are selected according to the relationship of each primer pair to the selected variants to be detected.

6-9. (canceled)

10. A kit for detecting in a sample the presence or absence of nucleotide variants at polymorphic sites in the gene encoding cytochrome P450-2C19, said variants selected from the group consisting CYP2C19*2, CYP2C19*3, CYP2C19*4, CYP2C19*5, CYP2C19*6, CYP2C19*7 and CYP2C19*8, said kit comprising a set of at least two tagged allele specific extension primers wherein each tagged allele specific extension primer has a 3′-end hybridizing portion including a 3′ terminal nucleotide being either complementary to a suspected variant nucleotide or to the corresponding wild type nucleotide of one of the polymorphic sites, and a 5′-end tag portion complementary to a corresponding anti-tag sequence, and wherein the 3′-end hybridizing portions of the at least two tagged allele-specific extension primers each comprise a sequence selected from the group consisting of nucleotides from position 25 to the 3′terminal nucleotide of SEQ ID NO: 12 to SEQ ID NO: 25.

11. The kit of claim 10 further comprising a set of PCR amplification primers for amplifying regions of DNA containing the polymorphic sites, said set selected from the group of primer pairs consisting of: SEQ ID NO: 2 and SEQ ID NO: 3, SEQ ID NO: 4 and SEQ ID NO: 5, SEQ ID NO: 6 and SEQ ID NO: 7, SEQ ID NO: 8 and SEQ ID NO: 9 and SEQ ID NO: 10 and SEQ ID NO: 11, wherein the PCR primer pairs are selected according to the relationship of each primer pair to the selected variants to be detected.

12. The kit of claim 10 further comprising a set of anti-tags.

13. The kit of claim 12 wherein the set of anti-tags are coupled to a solid support.

14-15. (canceled)

16. The method of claim 2 wherein the at least two tagged allele specific extension primers each comprise a sequence selected from the group of sequences consisting of SEQ ID NO: 12 to SEQ ID NO: 25.

17. The kit of claim 10 wherein the 5′-end tag portions of the at least two tagged allele specific primers each comprise a sequence selected from the group of sequences consisting of nucleotides from position 1 to 24 of SEQ ID NO: 12 to SEQ ID NO: 25, and wherein the sequence of each 5′-end tag portion is unique.

18. The kit of claim 17 wherein the at least two tagged allele specific primers are selected from the group of sequences consisting of SEQ ID NO: 12 to SEQ ID NO: 25.

19. The kit of claim 13 wherein the solid support is selected from the group consisting of beads, spectrally coded beads, and a chip based microarray.

20. A composition comprising a plurality of polynucleotide primers for use in detecting the presence of absence of nucleotide variants at polymorphic sites in the gene encoding cytochrome P450-2C19, said variants selected from the group consisting of CYP2C19*2, CYP2C19*3, CYP2C19*4, CYP2C19*5, CYP2C19*6, CYP2C19*7 and CYP2C19*8, wherein the plurality of primers comprises oligonucleotides having sequences set forth by the nucleotides from position 25 to the 3′ terminal nucleotide of SEQ ID NO: 12 to SEQ ID NO: 25, or the complete complements thereof.

21. The composition of claim 20 wherein the plurality of primers comprises oligonucleotides having sequences set forth by SEQ ID NO: 12 to SEQ ID NO: 25 or the complete complements thereof.

22. A combination comprising the composition of claim 21 wherein the plurality of primers consists of oligonucleotides having sequences set forth by SEQ ID NO: 12 to SEQ ID NO: 25, and further comprising a set of anti-tags having sequences complementary to nucleotides 1-24 of SEQ ID NO: 12 to SEQ ID NO: 25.

23. The combination of claim 22 wherein each anti-tag is attached to a spectrally coded bead specific for the anti-tag.

24. An improved method for simultaneously detecting in a sample the presence or absence of nucleotide variants at polymorphic sites in the gene encoding cytochrome P450-2C19, said variants selected from the group consisting of CYP2C19*2, CYP2C19*3, CYP2C19*4, CYP2C19*5, CYP2C19*6, CYP2C19*7 and CYP2C19*8, wherein the improvement comprises simultaneously identifying the presence or absence of variant nucleotides via allele-specific primer extension using a set of primers having the sequences set forth by SEQ ID NO: 12 to SEQ ID NO: 25.


SEQUENCE LISTING (25 sequences)

SEQ ID NO: 1 - DNA, 1473 bp, Homo sapiens
  ref <301>: AUTHORS: Koukouritaki,S.B., Manro,J.R., Marsh,S.A., Stevens,J.C., Rettie,A.E., McCarver,D.G. and Hines,R.N.
  ref <302>: TITLE: Developmental expression of human hepatic CYP2C9 and CYP2C19
  ref <303>: JOURNAL: Journal of Pharmacology and Experimental Therapeutics
  ref <304>: VOLUME: 308
  ref <305>: ISSUE: 3
  ref <306>: PAGES: 965-974
  ref <307>: DATE: 2004-03
  ref <308>: DATABASE ACCESSION NUMBER: NM_000769
  ref <309>: DATABASE ENTRY DATE: 2004-07-15
  ref <313>: RELEVANT RESIDUES: (1)..(1473)
  ref <308>: DATABASE ACCESSION NUMBER: NM_000769
  ref <309>: DATABASE ENTRY DATE: 2004-07-15
  ref <313>: RELEVANT RESIDUES: (1)..(1473)
atggatccttttgtggtccttgtgctctgtctctcatgtttgcttctcctttcaatctggagacagagctctgggagaggaaaactccctcctggccccactcctctcccagtgattggaaatatcctacagatagatattaaggatgtcagcaaatccttaaccaatctctcaaaaatctatggccctgtgttcactctgtattttggcctggaacgcatggtggtgctgcatggatatgaagtggtgaaggaagccctgattgatcttggagaggagttttctggaagaggccatttcccactggctgaaagagctaacagaggatttggaatcgttttcagcaatggaaagagatggaaggagatccggcgtttctccctcatgacgctgcggaattttgggatggggaagaggagcattgaggaccgtgttcaagaggaagcccgctgccttgtggaggagttgagaaaaaccaaggcttcaccctgtgatcccactttcatcctgggctgtgctccctgcaatgtgatctgctccattattttccagaaacgtttcgattataaagatcagcaatttcttaacttgatggaaaaattgaatgaaaacatcaggattgtaagcaccccctggatccagatatgcaataattttcccactatcattgattatttcccgggaacccataacaaattacttaaaaaccttgcttttatggaaagtgatattttggagaaagtaaaagaacaccaagaatcgatggacatcaacaaccctcgggactttattgattgcttcctgatcaaaatggagaaggaaaagcaaaaccaacagtctgaattcactattgaaaacttggtaatcactgcagctgacttacttggagctgggacagagacaacaagcacaaccctgagatatgctctccttctcctgctgaagcacccagaggtcacagctaaagtccaggaagagattgaacgtgtcattggcagaaaccggagcccctgcatgcaggacaggggccacatgccctacacagatgctgtggtgcacgaggtccagagatacatcgacctcatccccaccagcctgccccatgcagtgacctgtgacgttaaattcagaaactacctcattcccaagggcacaaccatattaacttccctcacttctgtgctacatgacaacaaagaatttcccaacccagagatgtttgaccctcgtcactttctggatgaaggtggaaattttaagaaaagtaactacttcatgcctttctcagcaggaaaacggatttgtgtgggagagggcctggcccgcatggagctgtttttattcctgaccttcattttacagaactttaacctgaaatctctgattgacccaaaggaccttgacacaactcctgttgtcaatggatttgcttctgtcccgcccttctatcagctgtgcttcattcctgtctga

SEQ ID NO: 2 - DNA, 28 bp, Artificial
  Forward Primer
actttatccatcaaagaggcacacacac

SEQ ID NO: 3 - DNA, 32 bp, Artificial
  Reverse Primer
gcatacttacattggttaaggatttgctgaca

SEQ ID NO: 4 - DNA, 26 bp, Artificial
  Forward Primer
gatggggaggatggaaaacagactag

SEQ ID NO: 5 - DNA, 30 bp, Artificial
  Reverse Primer
gtccagaaaggtcagtgatagagagtatgt

SEQ ID NO: 6 - DNA, 36 bp, Artificial
  Forward Primer
attatttaaccagctaggctgtaattgttaattcga

SEQ ID NO: 7 - DNA, 30 bp, Artificial
  Reverse Primer
tcaaaaatgtacttcagggcttggtcaata

SEQ ID NO: 8 - DNA, 31 bp, Artificial
  Forward Primer
caaccagagcttggcatattgtatctatacc

SEQ ID NO: 9 - DNA, 36 bp, Artificial
  Reverse Primer
gttaaacatccgtagtaaacacaaaactagtcaatg

SEQ ID NO: 10 - DNA, 32 bp, Artificial
  Forward Primer
catctatctactcatccctcctatgattcacc

SEQ ID NO: 11 - DNA, 31 bp, Artificial
  Reverse Primer
ggtcagaagaagcatcacagatagtgaaatt

SEQ ID NO: 12 - DNA, 46 bp, Artificial
  ASPE Primer
ctttctcatactttcaactaatttaagtaatttgttatgggttccc

SEQ ID NO: 13 - DNA, 46 bp, Artificial
  ASPE Primer
aatttcttctctttctttcacaataagtaatttgttatgggttcct

SEQ ID NO: 14 - DNA, 44 bp, Artificial
  ASPE Primer
taacataacttcttcattattcacggattgtaagcaccccctgg

SEQ ID NO: 15 - DNA, 44 bp, Artificial
  ASPE Primer
acactcatttaacactatttcattggattgtaagcaccccctga

SEQ ID NO: 16 - DNA, 44 bp, Artificial
  ASPE Primer
aatatacatctcaaacatttacacaggaccacaaaaggatccat

SEQ ID NO: 17 - DNA, 44 bp, Artificial
  ASPE Primer
acttacaataactactaatactctaggaccacaaaaggatccac

SEQ ID NO: 18 - DNA, 48 bp, Artificial
  ASPE Primer
tctcaacatataactcatttcttacaggccctctcccacacaaatccg

SEQ ID NO: 19 - DNA, 48 bp, Artificial
  ASPE Primer
cataatcaatttcaactttctactcaggccctctcccacacaaatcca

SEQ ID NO: 20 - DNA, 44 bp, Artificial
  ASPE Primer
tcttactaatttcaatactcttaccttccccatcccaaaattcc

SEQ ID NO: 21 - DNA, 44 bp, Artificial
  ASPE Primer
acacttatctttcaattcaattaccttccccatcccaaaattct

SEQ ID NO: 22 - DNA, 46 bp, Artificial
  ASPE Primer
atactaaacaattctctttactactcctgatcaaaatggagaaggt

SEQ ID NO: 23 - DNA, 46 bp, Artificial
  ASPE Primer
acaatatacatcacttaaactttctcctgatcaaaatggagaagga

SEQ ID NO: 24 - DNA, 44 bp, Artificial
  ASPE Primer
ctcaattactacatacatttaacaaaacgccggatctccttcca

SEQ ID NO: 25 - DNA, 44 bp, Artificial
  ASPE Primer
ctaactataatcttcatacacataaaacgccggatctccttccg